(19) United States
(12) Patent Application Publication
(10) Pub. No.: US 2024/0066302 A1
(43) Pub. Date: Feb. 29, 2024

(54) ADAPTIVE NEURAL INTERFACE

(71) Applicant: Ian Loren Halpern, Mill Valley, CA (US)

(72) Inventor: Ian Loren Halpern, Mill Valley, CA (US)

(21) Appl. No.: 18/118,666

(22) Filed: Mar. 7, 2023

Related U.S. Application Data
(60) Provisional application No. 63/317,530, filed on Mar. 7, 2022.

(51) Int. Cl. A61N 1/36; A61N 1/02; A61N 1/372; A61N 1/378

(52) U.S. Cl. CPC A61N 1/36135; A61N 1/025; A61N 1/37229; A61N 1/37235; A61N 1/3787

(57) ABSTRACT

A method of treating a neurological condition in a medical patient includes implanting first and second sets of leads at first and second target tissue sites, receiving first and second signals from the first and second sets of leads, determining that a parameter of the first signal satisfies a condition, determining that a parameter of the second signal does not satisfy the condition, and removing only the second set of leads from the medical patient. Each lead comprises one electrode or more than one electrodes spaced apart from each other along a length of each lead. The first signal is indicative of electrical activity at the first target tissue site during an event, and the second signal is indicative of electrical activity at the second target tissue site during the event.

### CROSS-REFERENCE TO RELATED APPLICATIONS

This application claims the benefit of priority from U.S. Provisional No. 63/317,530, filed Mar. 7, 2022, which is expressly incorporated by reference in its entirety. This application also incorporates by reference in their entireties U.S. application Ser. No. 15/192,905, filed Jun. 24, 2016 and U.S. Provisional No. 62/183,867, filed Jun. 24, 2015.

### STATEMENT REGARDING FEDERALLY SPONSORED R&D

This invention was made with government support under Department of Defense Contract/Federal Identifier Number W911NF-17-C-0057 awarded by the Department of Defense of the United States of America. The U.S. government may have certain rights in the invention.

### BACKGROUND

Various devices are configured to interface with the nervous system, and some may be implanted therein. However, implanted interfaces may require that a patient remain within a hospital setting during the entire implantation period. It would be useful, therefore, for a neural interface to be able to be left within a patient for hours, days, or weeks, while enabling the patient to participate in activities outside of a hospital setting. It would also be useful to use data collected from such long-term implantation periods to better understand, model, and treat any underlying conditions or health problems experienced by the medical patient during such periods.

### SUMMARY

Some aspects of the present invention relate to devices that conform to and interface with the nervous system and other forms of biological tissue. More particularly, some embodiments of the invention relate to device systems that can be implanted within tissue or assembled to the external surfaces of tissue to monitor or stimulate the nervous system upon the desired research or clinical purpose.

Disclosed herein are device systems, devices, and methods for modulating tissue, conforming to biological tissue, remaining stable adjacent biological tissue, reducing the irritation of tissue, and wirelessly sending and receiving power and communication through tissue. Devices can include in some embodiments components movable with respect to each other to reposition the system. Devices can include in some embodiments movable parts or structures that enable conformance to three-dimensional surfaces of tissue. In other embodiments, the devices are configured to conform to adjacent tissue and minimize the irritation of tissue, the generation of heat, and the restriction of movement of tissue. In some embodiments, one or more devices are configured at a target substrate. In other embodiments, two or more devices are configured adjacent separate target substrates. In some embodiments, devices are positioned to simultaneously communicate with another device. In some embodiments, the devices are positioned without the alignment of their antennae. In some embodiments, the devices are positioned with alignment of the antennae.

In still other embodiments, two or more devices are configured relative to one another to communicate through wires. In still other embodiments, the devices are configured to wirelessly transfer power and data. The devices can include for example a wireless antenna and an inductive coil for the wireless transfer of power. In other embodiments, aligned devices have multiple wireless communication systems. In some embodiments, the aligned devices have an inductive power coil system that is also used for wireless communication. In some embodiments, the aligned devices have an inductive power coil system.

In some embodiments, the device system is configured to record or stimulate tissue. In other embodiments the scales of components of the device system are configured to interact with target tissue. In still other embodiments, the device system is configured to predict tissue behavior or modulate tissue. The modulation of tissue can include the detection, prediction and prevention of epileptic seizures or the treatment of bradykinesia, tremor, migraine, motion, pain, spasm, paralysis, obsessive compulsive disorder, depression, and other conditions. In some embodiments, the modulation of tissue can include the treatment of the impairment or loss of vision, hearing, balance, proprioception, speech, taste, motor control, and other conditions.

In some embodiments, the device system is capable of transmitting wireless signals through tissue. The device system can include in some embodiments housings that are transparent to electromagnetic radiation. The device system can also include shielding adjacent antennas. The device system can also include a locating element that aligns an external device with an implanted device. Also, the device system can include housings that are partially transparent to electromagnetic radiation. In some embodiments, an antenna array includes two or more antennas. In still other embodiments, a device is operably connected to a neural probe. In some embodiments, a worn device can relay information wirelessly to another device. In other embodiments, the wirelessly transmitted information can include neural signals, heart rhythms, muscle firing signals, images, video, and other physiological signals. In some embodiments, also disclosed herein is a neural interface. The neural interface can include a housing configured to be implanted at a location within tissue and proximate neural tissue. The interface can include an antenna. There can be an air gap between the one or more radio frequency transparent windows and the antenna. The antenna can transmit the neural data via an ultra-wideband protocol or other wireless protocol. The antenna or additional antennas or wireless communication mechanisms can also be used to send or receive stimulation commands. The antennas can be part of a closed-loop control system. The housing can also include a processor configured to compare the frequency and/or modulation of a signal of communication. In some embodiments, each antenna could be enclosed in its own discrete housing.

In some embodiments, the devices are powered through wires. In still other embodiments, devices are powered through inductive coils. The devices can transmit power to other devices through tissue.

In some embodiments, the devices communicate with one another through wires. In still other embodiments, the devices wirelessly communicate with one another. The devices can wirelessly communicate with one another through skin.

In some embodiments, the device system utilizes magnetic force to position individual devices. Individual devices can contain magnetic materials that attract them to other components containing magnetic materials. Individual devices can apply a magnetic force to one another to maintain an advantageous alignment. In still other embodiments, the device system utilizes wireless feedback to position individual devices. In still other embodiments, the device system utilizes mechanical adjustment to position individual devices. An advantageous alignment can include positioning for inductive power transmission and wireless data transmission. An advantageous alignment can also include maintaining the comfort of tissue, and displacing with tissue as it moves. An advantageous alignment can also reduce the increase in temperature of a device. An advantageous alignment can also prevent a device from displacing during use.

In some embodiments, the device system or individual devices can be positioned in a single location within tissue or distributed throughout multiple locations within tissue. In some embodiments, a device is not adjacent tissue. In still other embodiments, a device is assembled external to clothing worn on the body.

In some embodiments, a device can have a single movable part or structure, enabling it to displace. The displacement can allow the device to conform to tissue. A device can also include no less than two and no more than twenty movable parts or structures, enabling it to displace.

In some embodiments, a device system or an individual device can contain one or more movable parts or structures aligned orthogonal to other movable parts or structures.

In some embodiments, a device can have two or more movable parts or structures orthogonal to two or more movable parts or structures.

In some embodiments, a device can be positioned adjacent a first target substrate of tissue, and another device can be positioned adjacent a second target substrate. In some embodiments, a device can be positioned adjacent a target substrate of tissue, and another device can be positioned adjacent a separate target substrate of tissue. In some embodiments, the device system can have one or more devices implanted within tissue. In other embodiments, the device system can have one or more devices assembled to the outer surface of the body. In some embodiments, the device system can have one or more devices implanted within tissue, and one or more devices assembled to the outer surface of the body. In some embodiments, the device system can have devices assembled adjacent a single target substrate of tissue.

In some embodiments, devices can be positioned by magnetic force. In some embodiments, devices can be partially held in position by adjacent tissue. In some embodiments, devices can be partially held in position by osseo-integration. In some embodiments, devices can be partially held in position by screws. In some embodiments, devices can be partially held in position by sutures. In some embodiments, devices can be held in position by mechanical locks. In some embodiments, devices can be partially held by clothing or worn components.

In some embodiments, the device system or individual devices can contain geometry and mechanisms capable of accelerating the dissipation of heat.

In some embodiments, protrusions or external guards are used to protect against excessive displacement of panels. In some embodiments, protrusions or external guards are used to protect against excessive displacement of movable parts or structures. The protrusions or external guards can prevent devices from dislodging from tissue, collapsing, or hyper-extending in a non-useful displacement.

In some embodiments, an assistive device is used to place and remove a device. In some embodiments, an assistive device can lower the force required to assemble or remove a device. In some embodiments, an assistive device sequentially removes sections adjacent movable parts or structures. Sequentially removing sections can result in a more gentle removal of a device from tissue rather than in an uncomfortable and or painful process.

In some embodiments, the device system includes a holder for devices. In some embodiments, a holder provides a protected position for a device. In some embodiments, the holder maintains the shape of a device in a resting state, preventing stress and strain on the movable parts or structures. In some embodiments, the holder maintains the shape of a device in a resting state, preventing stress relaxation and creep within the movable parts or structures.

Also disclosed herein are specific anatomical locations for assembling conforming devices.

Also disclosed herein are specific non-tissue related locations for assembling conforming devices.

In some aspects, the techniques described herein relate to a method of treating a neurological condition in a medical patient, including: implanting a first set of leads at a first target tissue site within target tissue of the medical patient, wherein each lead includes one electrode or more than one electrodes spaced apart from each other along a length of each lead of the first set of leads; implanting a second set of leads at a second target tissue site within the target tissue of the medical patient, wherein each lead includes one electrode or more than one electrodes spaced apart from each other along a length of each lead of the second set of leads; receiving a first signal via the first set of leads, the first signal indicative of electrical activity at the first target tissue site during an event; receiving a second signal via the second set of leads, the second signal indicative of electrical activity at the second target tissue site during the event; determining that a parameter of the first signal satisfies a condition; determining that a parameter of the second signal does not satisfy the condition; and removing only the second set of leads from the medical patient.

In some aspects, the techniques described herein relate to a method, further including sending a stimulating signal to the first target tissue site via the first set of leads.

In some aspects, the techniques described herein relate to a method, wherein receiving the first signal via the first lead includes receiving the first signal via the first lead continuously or intermittently over a predetermined data collection period of minutes, hours, days, weeks, or months.

In some aspects, the techniques described herein relate to a method, wherein the target tissue includes brain tissue, nerve tissue, cardiac tissue, or bone.

In some aspects, the techniques described herein relate to a method, wherein the parameter of the first signal includes a Voltage level, a current level, a frequency, or an impedance.

In some aspects, the techniques described herein relate to a method, wherein the first set of leads includes a single lead, about 10 leads, about 30 leads, about 50 leads, about 100 leads, about 1000 leads, about 2,500 leads, or more than 1000 leads.

In some aspects, the techniques described herein relate to a method, wherein implanting the first set of leads at the first target tissue site includes implanting the first set of leads at the first tissue site at a first depth, wherein implanting the second set of leads at the second target tissue site includes implanting the second set of leads at a second depth within the target tissue, and wherein the first depth is different than the second depth.

In some aspects, the techniques described herein relate to a method, wherein the event includes electrical activity related to a seizure, an epileptic seizure, seizure onset, therapy resistant depression, vision restoration, hearing restoration, speech restoration, motor control restoration, sleep dysfunction, or Alzheimer's disease.

In some aspects, the techniques described herein relate to a method, wherein the first set of leads includes a wireless transmitter or transceiver configured to wirelessly transmit the first signal to a wireless receiver or transceiver.

In some aspects, the techniques described herein relate to a method, wherein receiving the first signal includes wirelessly receiving the first signal from a wireless transmitter coupled to the first set of leads.

In some aspects, the techniques described herein relate to a method, further including resecting at least a portion of the target tissue at the first or second target tissue site.

In some aspects, the techniques described herein relate to a method, wherein determining that a parameter of the first signal satisfies a condition includes utilizing a machine learning algorithm, deep learning, a neural network, or artificial intelligence.

In some aspects, the techniques described herein relate to a method, further including determining a stimulation based upon a detected condition utilizing a machine learning algorithm, deep learning, a neural network, or artificial intelligence.

In some aspects, the techniques described herein relate to a medical headset configured to treat a neurological condition in a medical patient, including: a headgear configured to be worn on the head of a medical patient and configured to be positioned or located with respect to an implant without the use of magnets; a wireless receiver or transceiver supported by the headgear and configured to receive a wireless signal from a wireless transmitter coupled to a first set of leads implanted at a first target tissue site within target tissue of the medical patient; a power source, supported by the headgear and configured to: (i) provide power to the wireless receiver or transceiver, and (ii) wirelessly provide power to the implant; a processor, coupled to the wireless receiver and configured to store neural activity data corresponding to the wireless signal in a data store; and the data store.

In some aspects, the techniques described herein relate to a medical headset, further including an interface for transferring the neural activity data to a computing system.

In some aspects, the techniques described herein relate to a medical headset, wherein the interface includes a USB port, a wireless transceiver, a SIM slot, or a memory card slot.

In some aspects, the techniques described herein relate to a medical headset, wherein the power source includes a battery.

In some aspects, the techniques described herein relate to a medical headset, wherein the data store includes a removable memory device configured to be removed from the medical headset and transported to a caregiver, or a machine learning algorithm developer.

In some aspects, the techniques described herein relate to a medical headset, wherein the medical headset further includes an adjustable mechanical engagement of an applied force source and mechanical detents in at least two dimensions, wherein the adjustable mechanical engagement enables fine tuning and adjustment of the position of the wireless receiver or transceiver with respect to an implanted wireless transmitter or transceiver of the implant.

In some aspects, the techniques described herein relate to a medical headset, further including a sensor, the sensor including one or more of a camera, an accelerometer, a thermometer, a humidity sensor, a perspiration sensor, a gyroscope, a switch, a microphone, a light sensor, or a GPS, wherein the sensor is configured to generate a signal associated with or an image of the medical patient, and wherein the processor is further configured to determine a clinical event based on the signal.

### BRIEF DESCRIPTION OF THE DRAWINGS

The embodiments described herein are illustrated by way of example, and not by way of limitation, in the figures of the accompanying drawings in which like references indicate similar elements.

FIG. 1 shows an isometric view of a device implanted within tissue connected to another device, according to some embodiments.

FIG. 1A shows an isometric view of a device implanted within tissue connected to another device and an enlarged isometric view of a device implanted within tissue, according to some embodiments.

FIG. 1B shows a side view and bottom view of a device implanted within tissue for recording and stimulation purposes.

FIG. 1C shows a side view and construction of a device for recording from and stimulating tissue.

FIG. 1D shows a view of a device anchored to tissue.

FIG. 2 shows an isometric view of a device implanted for interfacing with tissue.

FIG. 3 shows an isometric view of a device system containing an implanted device and a worn device.

FIGS. 3A and 3C show isometric views of a device system assembled to tissue.

FIG. 3B shows a cross sectional view of a portion of a device system assembled to tissue.

FIG. 4 illustrates a block diagram of a wireless device system, according to some embodiments.

FIG. 5 illustrates a computer system that can be configured for use with a wireless neural data system, according to some embodiments.

### DETAILED DESCRIPTION

Biological sensors are implanted within the bodies of animals and humans to study, record, stimulate, and treat various anatomies. Typically, this occurs within animal research of a variety of fields (e.g., neurological disorders and basic nervous system function) as well as clinical diagnosis, prediction, and therapy (e.g., epilepsy).

Biological sensors are implanted for a variety of reasons, including wirelessly transmitting signals into and out of the body, through a variety of methods including radio frequency, ultrasonic, and optical transmissions. Radio frequency communication is the most pervasive form of wirelessly communicating with devices implanted within the body. It is advantageous to transmit large amounts of data into or out of the body, some non-limiting examples of this need is the recording of neurons or muscle cell firing, ion channel activity, or the sending of specific commands for stimulation or ablation. The firing of a neuron produces a distinct voltage signature over time that requires a detailed analog recording to identify. These analog recordings over long periods of time require large amounts of data to be recorded and transmitted. Transmitting at a high total data rate out of the body is advantageous as it increases the number of neuron firing events that can be identified over time.

Unfortunately, wireless transmissions are attenuated by tissue, limiting the amount of data transmitted between an implanted device and external devices. Attempts to work around this challenge are problematic and include increasing the number of implanted devices, as well as reducing tissue thicknesses to reduce signal attenuation. Reducing tissue thicknesses is time consuming during surgery and damaging or irreversible for most organisms. Increasing the number of implanted devices has proven difficult due to wireless transmissions of individual implants disrupting one another. Increasing the number of implants also increases the invasiveness of the procedure and device, increasing the risk of unsuccessful results for the implant due to infection or other surgical complications. Another significant challenge for implanted devices is providing them with power. Powering a dispersed network of implants provides many additional challenges to a device system, and connecting nodes of an implant network with cables to a single power source increases the difficulty and delicacy of implantation and aides the spread of infection from one area to another within a body. Alignment of external components with implanted antennas can also be challenging. These limitations prevent the implantation of wireless chronic neural interfaces in a wide variety of situations. This reduces the amount of data acquired, limiting the study of anatomy, and ultimately limits current and future therapies.

Accordingly, in some embodiments, disclosed herein is a wireless device system that increases the amount of data that can be transmitted into and out of the body within a compact device system. In some embodiments, the system may have one or more antennas operating on the same or overlapping frequency spectrum arranged in a shielded array to prevent interference with one another while increasing the total amount of data transmitted into and out of the body. It can also be advantageous to place arrays in the body in areas with thinner tissue; in one embodiment, this may be the scalp. In some embodiments, the antenna arrays may be configured with at least one other antenna operating on another frequency spectrum. As an example, this would enable one frequency for communicating with basic functions of an implanted device and another frequency spectrum for transmitting large amounts of information into and out of the body.

In some embodiments, tissue can be altered to increase the strength or reduce attenuation of wireless communication between devices. In other embodiments, tissue can be altered for positioning of a device that increases the strength or reduces attenuation of wireless communication with another device. In some embodiments, the tissue is a component of the wireless device as it alters the performance based on its thickness and density. The wireless device system's performance is affected by its integration with tissue. The tissue thickness, alignment of implanted and external antennas and the distance between implanted and external antennas, contributes to reliable communication and electromagnetic radiation emitted through tissue not contained by the device system. Designing the wireless device system for specific tissue thicknesses and anatomical locations can increase the system performance, and limits the electromagnetic radiation emitted through tissue outside the body. Limiting the electromagnetic radiation emitted through tissue outside the body and the device system can in some cases increase safety, reduces the possibility of interference with other wireless devices, and satisfies the requirements of regulatory organizations. In some embodiments, the system can be designed and configured for tissue thicknesses of 5 mm to 25 mm, such as no more than about 6 mm, 7 mm, 8 mm, 9 mm, 10 mm, 11 mm, 12 mm, 13 mm, 14, mm, 15 mm, 17 mm, 19 mm, 21 mm, or 24 mm. In still other embodiments, the system can be designed and configured for tissue thicknesses of 1 mm to 5 mm, such as no more than about 2 mm, 3 mm, or 4 mm. In some other embodiments, the system can be designed for tissue thicknesses of 25 mm to 45 mm, such as no more than about 28 mm, 32 mm, 36 mm, 40 mm, or 44 mm.

In some embodiments, disclosed herein is a device system that can integrate with various types of neural interfaces that act as recording or stimulation electrodes, optical fibers, or as hollow tubes for media, e.g., fluid delivery. In still other embodiments, advantageous configurations for various treatment modalities including recording, stimulating, magnetic stimulation, magnetic monitoring, fluid delivery, temperature control, optical stimulation, optical monitoring, video monitoring, and chemical irrigation of neural tissue. In some embodiments, the systems and methods can be utilized to monitor nerve firing to locate nerves, such as parasympathetic, sympathetic, afferent, and/or efferent nerves prior to or during a denervation proceeding, such as renal denervation, a cardiac EP study, vagal nerve ablation, and the like.

In some embodiments, the device system can also serve as a delivery device for a drug, such as an antithrombotic agent, an antibiotic, an anti-inflammatory, an anti-epileptic, viral vectors, or a chemotherapeutic agent, for example. In some embodiments, the device system can be implanted within any tissue within the body dependent upon the desired research or clinical result; including nervous, muscle, connective, epithelial, cardiac, lung, renal, gastrointestinal, and bone tissues.

Neuroscientists and clinicians need devices that monitor and treat nervous systems and other biological tissues from implanted and external positions. These devices need to adapt and conform to the spectrum of complex three-dimensional surfaces of tissues. These shapes can include approximately concave, approximately flat, and approximately convex, as well as many other three-dimensional shapes.

Neuroscientists and clinicians have a well-recognized need to wirelessly communicate with large groups of neurons and transmit wireless power through tissue. Available research devices struggle to demonstrate reliable wireless transmission of data and power.

Neural interface devices are inserted into tissue and assembled to tissue in a variety of ways including mechanical insertion, suturing to tissue, adhesive based assembly, and the use of magnetic materials. The most pervasive forms of assembly are the removal of bone, screwing of implanted devices into place, and the holding in place of devices external to the body by magnetic force. Maintaining external assembly of a device to a body requires a balance between a strong enough magnetic force of attraction, a low enough pressure applied to the skin, and the conformance of the device to the shape of the body to mechanically engage with it. Maintaining a close proximity and alignment between radio antennas and inductive coils adjacent tissue is a significant need of researchers. Currently available devices are assembled in relatively flat sections of tissue. Devices assembled to flat sections of tissue are more susceptible to dislodgment by shear forces than when assembled to three-dimensionally curved sections of tissue, which provide a mechanical engagement in one or more directions for the device.

Unfortunately, there are limited devices available that can wirelessly communicate data about the nervous system from beneath the skin, leading to a reliance on devices that protrude through the skin. The devices that are available for implantation under the skin either have a limited ability to record or stimulate the nervous system and thus are small in nature, or, are large, bulky, and do not confirm to tissue within or outside of the body. The variations in thickness of tissue and the movement of tissue pose significant challenges to maintaining alignment and proximity of antennas and inductive coils for assembled devices. Currently available neural interface devices are made for insertion of one or perhaps two units into the brain at deeper levels. There is a need for a three-dimensional constellation of numerous neural interface units distributed throughout deeper levels of the brain to create a constellation of continuous data to better understand neural mechanisms that span the brain. Importantly, implanted neural interfaces are expensive and any design or method that reduces cost would increase availability for research. These limitations prevent the implantation of chronic neural interfaces in a wide variety of situations. This reduces the amount of data acquired as well as limiting current and future therapies.

Accordingly, in some embodiments, disclosed herein is a device system and devices that provide unlimited degrees of freedom for placing, stabilizing, and removing devices that conform to the nervous system and other tissues.

In some embodiments, the device system or an individual device can interface with the nervous system to diagnosis and/or treat epilepsy, a movement disorder (e.g., Parkinson's Disease), a psychiatric disorder (e.g., clinical depression), the result of a stroke, Alzheimer's disease, a cognitive disorder, an anxiety disorder, an eating disorder, an addition or craving, restless leg syndrome, a sleep disorder, Tourette's syndrome, a stress disorder, coma, autism, a hearing disorder, a vision disorder, blindness, retinal degeneration, age related macular degeneration, cortical injury, optic nerve injury, dry eye syndrome, a balance disorder, a speech disorder, amblyopia, ageusia, anosmia, headaches, temporomandibular joint disorder, pain (e.g., phantom limb pain and chronic pain), urinary incontinence, erectile dysfunction, bone disease, arthritis, tendonitis, the result of ligament or tendon damage, loss of motor control, and paralysis (e.g., facial nerve paralysis and spinal paralysis). In some embodiments, the device system or an individual device can be used to provide control of a prosthetic such as a limb or an external computer.

In some embodiments, the device system or an individual device may wirelessly communicate with a system that is connected to a network or cloud of data. In other embodiments, the device system is connected to a biological interface to monitor tissue. In some other embodiments, the device system is connected to a biological interface to modulate tissue. In still other embodiments, the device system is connected to a biological interface to monitor and modulate tissue. In other embodiments, the biological interface can include an implantable camera.

In other embodiments, the device system or an individual device can study, diagnose, and/or treat cardiovascular conditions such as heart failure, rheumatic heart disease, hypertensive heart disease, ischemic heart disease, angina, coronary artery disease, cerebral vascular disease, stroke, atherosclerosis, cerebrovascular disease, cardiomyopathy, pericardial disease, valvular heart disease, inflammatory heart disease, congenital heart disease, and peripheral arterial disease.

In still other embodiments, the device system or an individual device can study, diagnose, and/or treat cancers, including leukemia, lymphoma, myeloma, bladder cancer, lung cancer, brain cancer, melanoma, breast cancer, non-Hodgkin lymphoma, cervical cancer, and ovarian cancer.

In other embodiments, the device system or an individual device can study, diagnose, and/or treat type 1 and type 2 diabetes. In some embodiments, the device system can include a biological interface to study, diagnose, and/or treat orthopedic conditions, including osteoarthritis, rheumatoid arthritis, bone fractures, lower back pain, neck pain, and a herniated disk.

In other embodiments, the device system or an individual device can study, diagnose, and/or treat eye conditions, including glaucoma, cataracts, age-related macular degeneration, amblyopia, diabetic retinopathy, retinal detachment, retinal tearing, and dry eye syndrome.

In still other embodiments, the device system or an individual device can study, diagnose, and/or treat hearing conditions, including hearing loss, Meniere's disease, malformation of the inner ear, autoimmune inner ear disease, tinnitus, and vertigo.

In other embodiments, the device system or an individual device can study, diagnose, and/or treat tactile disorders, including impaired sensitivity to pressure applied to the skin, elevated two-point discrimination thresholds (e.g., impaired spatial acuity), loss of vibratory sense, and deficits in proprioception.

In other embodiments, the device system or an individual device can study, diagnose, and/or treat taste, taste impairing conditions, smell, and smell impairing conditions.

In still other embodiments, the device system can be movably engaged within one, two, or more body tissues, regions, or organ systems including but not limited to the scalp, skin, muscle, bone, neural tissue, heart, lungs, trachea, bronchi, diaphragm, liver, pancreas, kidneys, bladder, urethra, spleen, esophagus, stomach, intestine, penis, testes, uterus, or ovary.

In some embodiments, provided is a closed loop control system for stimulating (or ablating) and monitoring neural activity. In some embodiments, systems and methods as disclosed herein can modulate neural tissue, and have a stimulatory or inhibitory effect. Neural tissue is specialized for the conduction of electrical impulses that convey information or instructions from one region of the body to another. About 98% of neural tissue is concentrated in the brain and spinal cord, which are the control centers for the nervous system. Neurons transmit signals as electrical charges which affect their cell membranes. A neuron has a cell body (soma) that contains a nucleus. The stimulus that results in the production of an electrical impulse usually affects the cell membrane of one of the dendrites, which then eventually travels along the length of an axon, which can be a meter long. Axons are often called nerve fibers with each ending at a synaptic terminal. Neuroglia are cells of the CNS (central nervous system) and PNS (peripheral nervous system) that support and protect the neurons. They provide the physical support for neural tissue by forming myelin sheaths, as well as maintaining the chemical composition of the tissue fluids and defending the tissue from infection. Schwann cells are specialized PNS cells that form myelin sheaths around neurons. Neurons (nerve cell) include a cell body that contains the nucleus and regulates the functioning of the neuron. Neurons also include axons that are cellular process (extension) that carry impulses away from the cell body. Neurons also include dendrites that are cellular process (extension) that carry impulses toward the cell body. A synapse is a space between axon of one neuron and the dendrite or cell body of the next neuron—transmits impulses from one neuron to the others. Neurotransmitters are chemicals released by axons and transmit impulses across synapses.

In still other embodiments, provided is a closed loop control system for stimulating and monitoring physiological activity. In other embodiments, systems and methods as disclosed herein can modulate tissue and organs, and have a stimulatory or inhibitory effect. A system could include a pair of antennas configured for stimulation and/or recording. The antennas can operate at high data rates. The pair of antennas could be partially, exclusively, or substantially entirely dedicated to sending recording signals out of the body (or to another location within the body). The pair of antennas can also be partially, exclusively, or substantially entirely dedicated to send stimulation signals to stimulate electrodes, end effectors delivering RF, microwave, electromagnetic, ultrasound, thermal, cryo, and/or other energy, fluid, etc. to antennas within the body or external to the body. This can advantageously allow for wireless closed loop control at high channel counts and data rates. In some embodiments, the system can be configured to record and/or stimulate about or at least about 100, 150, 250, 500, 1,000 channels, or even more. In other embodiments an inductive power coil can be used to send stimulation signals to stimulate electrodes, end effectors delivering RF, microwave, electromagnetic, ultrasound, thermal, cryo, and/or other energy, fluid, etc. to antennas within the body or external to the body.

In some embodiments, microfilaments are used to record and stimulate neural tissue. In still other embodiments it is advantageous that the approximate diameter of circular microfilaments for conducting electrical current is between 1 μm and 250 μm, such as no more than about 5 μm, 10 μm, 25 μm, 50 μm, or 75 μm. For electrical stimulation, larger sites up to 50 μm would be advantageous to achieve surface areas that meet useful stimulation current requirements without a coating. The approximate diameter of circular microfilaments for conducting or monitoring light is between is 0.1 μm to 250 μm, such as no more than about 25 μm, 50 μm, or 75 μm. The approximate diameter of circular microfilament tubes for delivering or circulating gases, fluids, and mixtures in some embodiments is between 1 μm to 100 μm, or no more than about 50 μm, 75 μm, 100 μm, or 150 μm. Microfilaments can also be placed within a packed geometry that allows for a tapering of the penetrating area cross sections to reduce the cross-sectional area and thus long term adverse neural tissue response. In some embodiments, the microfilaments can extend outward from the body's surface; these sites can be formed (e.g., bent or flattened) to provide desired functional characteristics. In some embodiments, the microfilaments are conductively assembled to larger electrode discs or other shapes including circles, ovals, ellipsoids, rhombi, for recording and stimulating neural tissue. In some embodiments, the approximate diameter of circular electrode discs for conducting electrical current is between 50 μm to 750 μm, such as no more than about 100 μm, 200 μm, 300 μm, 400 μm, 500 μm, 600 μm or 700 μm. In other embodiments, the approximate area of electrode shapes is 25 μm², 50 μm², 100 μm², 200 μm², 500 μm², 1000 μm², 2500 μm 2. In still other embodiments, the approximate area of electrode shapes is 0.01 mm², 0.0625 mm², 0.25 mm², 0.5 mm², 1.0 mm², 1.5 mm², 2.0 mm², 2.5 mm², 3.0 mm², 3.5 mm², or 4.0 mm². In other embodiments, the approximate area of electrode shapes is 5.0 mm², 10.0 mm², 20.0 mm², 30.0 mm², 50.0 mm², 60.0 mm², 70.0 mm², 80.0 mm², 90.0 mm², 100.0 mm², or 110.0 mm².

A microfilament array body can take multiple forms including penetrating structures with microfilament sites and joining sections to optimize placement within the nervous system. An approximate cross-sectional area of a penetrating array body in some embodiments is 0.049 mm² to 3.2 mm², preferably up to approximately 5.0 mm². In other embodiments, an approximate cross-sectional area of a penetrating body is 0.0025 mm² to 0.0225 mm², preferably up to approximately 0.05 mm². For large area coverage as in electrocorticography, larger body areas up to approximately 100 cm² or more would be advantageous to collect more data from the outer surface of a neural tissue section. Systems and methods as disclosed herein can be used or modified for use with neural arrays as disclosed, for example, in U.S. Pat. No. 9,095,267 to Halpern et al., which is hereby incorporated by reference in its entirety.

In some embodiments, the device system is movably assembled within a sealed connector assembly. In still other embodiments, the connector is movably assembled to another connector containing the wireless device system. In some embodiments, the wireless device system embedded in a connector assembly might be advantageous underwater, in corrosive environments, in low-visibility environments, in outer space, and in applications requiring a low force of connection. In other embodiments, the device system embedded within a wireless connector would be advantageous in blood, urine, hormones, and lymph. In still other embodiments, the device system would be advantageous in environments that limit the use of hands, including industrial, athletic, and military pursuits.

One advantage of an implantable wireless device in some embodiments is the wide range of materials and components available to improve implantation conditions and long-term performance of a device within a nervous system. The components of the device can be formed from titanium, niobium, gold, platinum, platinum iridium, iridium, carbon, stainless steel, steel, aluminum, conductive polymers, polymers, ceramics, organic materials, combinations of the foregoing, or any other materials.

In some embodiments, provided is a closed loop control system for stimulating and monitoring neural activity. To meet this objective, microfilaments are embedded in various body configurations to provide many system options for interacting with neural tissue. As an example, this would enable the data collected from an implanted device (or external source) to help guide the output of a second stimulating device. In some embodiments, data from an implanted device monitoring neural tissue could be used to predict physiological activity and determine stimulation commands to prevent or augment the predicted activity. In some embodiments, the neural tissue behavior recorded includes single units firing, multi-unit activity, local field potentials, micro scale local field potentials, electrocorticography, intracranial electroencephalography, intracranial stereo electroencephalography, sub scalp electroencephalography, and supra scalp electroencephalography.

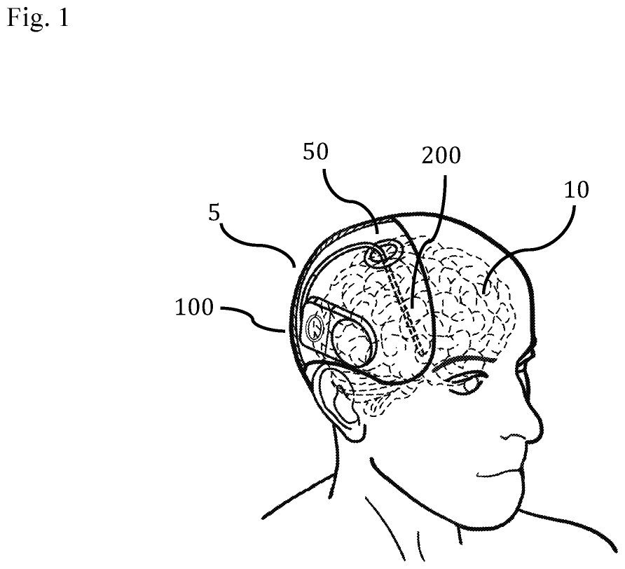

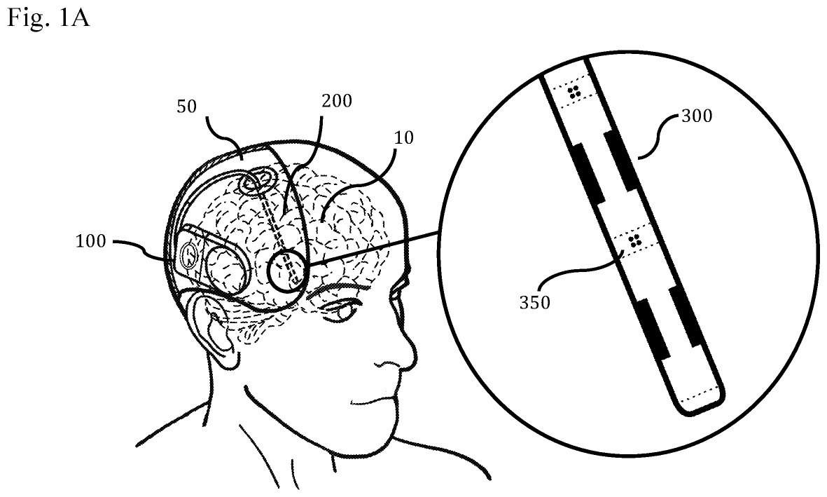

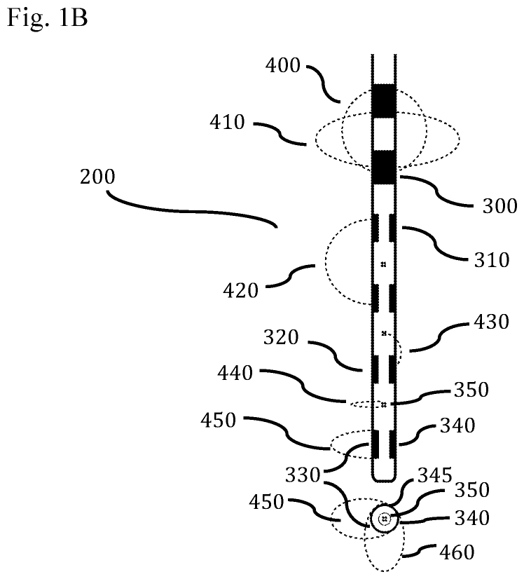

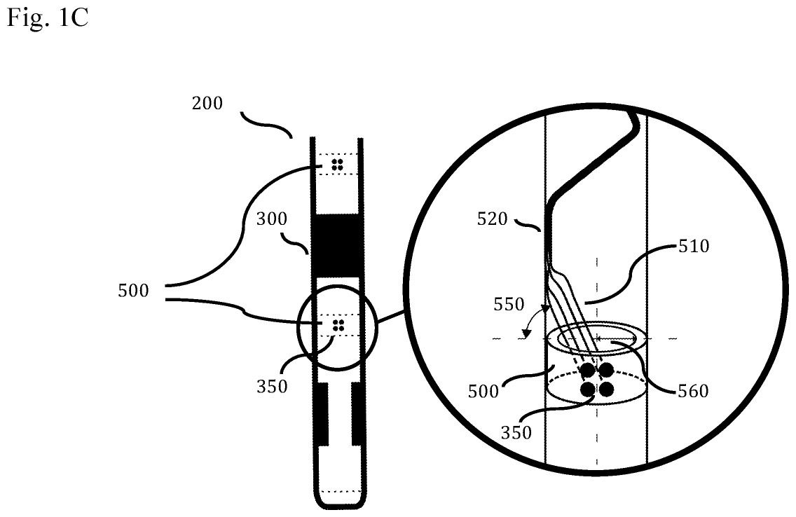

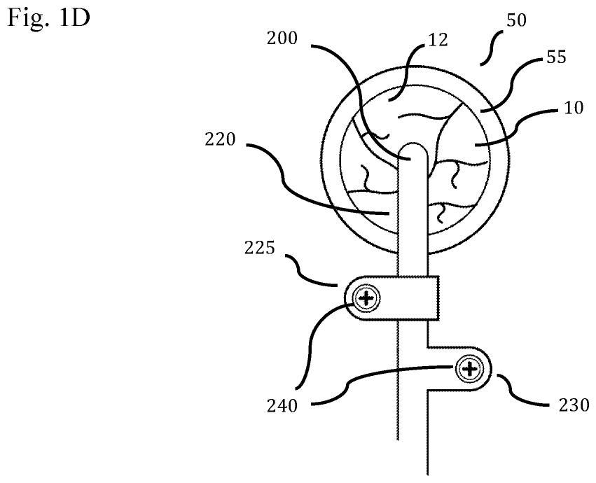

FIG. 1 shows an isometric view of a device system 5 including an implanted device 100 and a device 200 implanted in tissue 10. In some embodiments, one or more device 200 are assembled to the device 100. In other embodiments, two devices 100 are assembled to one or more device 100. In still other embodiments, three or four devices 200 are assembled to the device 100. In other embodiments, the device 200 is used for recording and stimulation. In some embodiments, the device 200 is used for recording tissue, e.g., electrical signals generated or conducted by or through the tissue 10. In some embodiments, the device 200 is used for stimulation of tissue. In other embodiments, the device 200 is positioned adjacent tissue 50.

In some embodiments, the implanted device 200 is placed at a location beneath the skin. In other embodiments, the implant is placed beneath organs. In still other embodiments, the bone is shaped to provide additional clearance for placement of the implant and increase the strength of wireless communication. In other embodiments, the device is placed within tissue at some location below the skin, such as, for example, adjacent the Intrinsic cardiac ganglia, Cardiac plexus, Cardiac ganglion, Nodose ganglion, Structure of superior cervical ganglion, Sympathetic ganglion, Stellate ganglion, Postganglionic sympathetic nerve fibers of neck and thorax, Peripheral ganglia, Sensory ganglia, Spinal ganglia, Auricular branch of the vagus (ABVN), Nucleus tractus solitarius (NTS), Dorsal medullary vagal system, Spinal cord, Peripheral neurons innervating the bladder, Peripheral and spinal neural circuitry of the lower urinary tract (LUT), Sensory and autonomic neurons that innervate the bladder body, trigone and proximal urethra, Vagal branches innervating the stomach, Nerves projecting to the intrapulmonary airways, Preganglionic parasympathetic nerves innervating the airways, Superior cervical ganglia (SCG), Hypoglossal nerve, Structure of parasympathetic ganglion, Enteric nervous system (ENS), ganglia of the ENS intrinsic to the gut wall, the Vagus nerve (VN), or in between any of the foregoing structures. In some embodiments, the internal device can be placed on an endothelial, mesothelial, or adventitial layer of tissue, for example, or under or within the epidermis, dermis, or hypodermis of the skin. In some embodiments, the device is placed within the skeleton, organs, nails, hair, ear, finger, hand, nose, nostril, eye, tongue, tooth, or teeth.

In some embodiments, the implanted device is placed at a location beneath the scalp. In other embodiments, the implant is placed beneath the cranium. In still other embodiments, the bone is shaped to provide additional clearance for placement of the implant and increase the strength of wireless communication. In other embodiments, the device is placed within tissue at some location below the skull/cranium, such as, for example, deep to the skull, dura mater, arachnoid, pia mater, or bridging veins, and/or superficial to, or in between any of the foregoing structures. In some embodiments, the internal device can be placed on an endothelial, mesothelial, or adventitial layer of tissue, for example, or under or within the epidermis, dermis, or hypodermis of the skin.

FIG. 1A shows an isometric view of a device 200 implanted in tissue 10. In some embodiments, the device 200 has shaft diameter between about 0.1 mm and 2.0 mm, such as no more than about 0.25 mm, 0.5 mm, 0.75 mm, 1.0 mm, 1.25 mm, 1.50 mm, or 1.75 mm. In some embodiments, the device 200 has electrodes 300 and 350 for recording neural activity. In some embodiments, the device 200 has electrodes 300 and 350 for recording electroencephalography (EEG). In some embodiments, the device 200 has electrodes 300 and 350 for recording local field potentials (LFPs). In some embodiments, the device 200 has electrodes 300 and 350 for recording LFP, multi-unit activity, and individual neurons. In some embodiments, electrodes 300 and 350 can stimulate tissue. In some embodiments, the electrode sites 300 have a diameter of 0.5 mm to 2.0 mm. In some embodiments the electrode sites 300 have a length of 0.25 mm to 3.0 mm. In some embodiments, electrode sites 350 have a diameter between about 0.005 mm and 0.6 mm, such as no more than about 0.012 mm, 0.025 mm, 0.05 mm, 0.1 mm, 0.2 mm, 0.25 mm, 0.3 mm, 0.4 mm, or 0.5 mm.

FIG. 1B shows a side view and a bottom view of a device 200. In some embodiments, the electrodes 350 are grouped in patterns. In other embodiments, the electrodes 350 are grouped in patterns of two, three, or four. In other embodiments, the electrodes 350 are grouped in patterns with spacing between electrodes between about 0.005 mm and 0.75 mm, such as no more than about 0.01 mm, 0.05 mm, 0.1 mm, 0.2 mm, 0.3 mm, 0.5 mm, 0.6 mm, or 0.7 mm. In some embodiments the electrodes 300 are separated into segments of two sections 310 and 320. In some embodiments the electrodes 300 are separated into segments of two, three, or more sections. Using electrodes 300, 310, 320, 330, or 350 individually or in combination with one another to stimulate tissue can produce different stimulation profiles for activating tissue volumes dependent upon stimulation parameters including timing, coordinated timing, frequency, duration, and amplitude. Each combination of stimulation parameters produces different activated tissue volumes, including stimulation profiles 400, 410, 420, 430, 440, and 450.

In some embodiments, device 200 can have a length of for example, between about 1 cm and 10 cm, such as no more than about 2 cm, 4 cm, 6 cm, or 8 cm. In some embodiments, device 200 can have a length of for example, between about 0.1 cm and 30 cm, such as no more than about 0.5 cm, 1 cm, 2 cm, 5 cm, 10 cm, 15 cm, 20 cm, or 25 cm. In some embodiments, device 200 has an axis significantly longer than its orthogonal axis. In some embodiments device 200 has recording sites. In some embodiments, device 200 has stimulation sites. In some embodiments, device 200 has recording electrodes 300. In still other embodiments, the device 200 has recording electrodes 350. In some embodiments, the electrodes 300 record and stimulate. In some embodiments, the electrodes 350 record and stimulate. In still other embodiments, the device 200 has stimulating electrodes 300. In some embodiments, the device 200 has recording electrodes 350. In other embodiments, the electrodes 300 are positioned to record and stimulate in a coordinated way. In other embodiments, the electrodes 350 are positioned to record and stimulate in a coordinated way. In still other embodiments, the electrodes 300 and 350 are positioned to optimize stimulation. In still other embodiments, the electrodes 300 and 350 are positioned to optimize recording.

In other embodiments, the electrodes 350 have spacing between about 0.025 mm and 0.5 mm, such as no more than about 0.05 mm, 0.075 mm, 0.1 mm, 0.125 mm, 0.15 mm, 0.2 mm, 0.25 mm, 0.3 mm, 0.35 mm, 0.4 mm or 0.45 mm. In other embodiments, the electrodes 350 have spacing between about 0.5 mm and 2.5 mm, such as no more than about 0.75 mm, 1.0 mm, 1.5 mm, 1.75 mm, 2.0 mm, or 2.25 mm. In some embodiments, electrodes 350 are disks, cylinders, tubes, film, or assemblies of tubes and cylinders. In some embodiments the electrode sites are mechanically deformed, patterned, or coated to improve performance by increasing surface area. In other embodiments the electrodes 350 have more than 4 grouped electrodes. In some embodiments, the electrodes 350 are arranged in non-linear, spiral, radial, geometric, or asymmetric patterns. In some embodiments the electrodes 350 have one, two or three, electrodes.

FIG. 1C shows a side view of a device 200 with a construction element 500. In some embodiments, the device 200 has a movable part or structure such as electrodes 300 and a movable part or structure 500. In some embodiments, the construction element 500 is spaced away from electrodes 300 or other electrodes between about 0.5 mm and 2.5 mm, such as no more than about 0.75 mm, 1.0 mm, 1.5 mm, 1.75 mm, 2.0 mm, or 2.25 mm. In some embodiments, the construction element 500 is arranged for assembly within device 200. In some embodiments construction element 500 arranges wires connected to electrodes 350 into a single group 510. In other embodiments construction element 500 winds wires connected to electrodes 350 into a single group with wires adjacent one another in a group 520. In other embodiments group 520 braids wires together. In other embodiments group 520 braids wires together in a helical pattern. In some embodiments group 510 is arranged in a helical pattern within device 200. In some embodiments group 520 is arranged in a helical pattern within device 200. In some embodiments construction element 500 arranges wires connected to electrodes 350 to exit at an angle 550. In some embodiments the angle 550 is between about 5 degrees and 45 degrees such as no more than about 10 degrees, 20 degrees, 30 degrees, or 40 degrees. In some embodiments the distance 560 is between about 0.1 mm and 1.75 mm such as no more than about 0.25 mm, 0.5 mm, 0.75 mm, 1.0 mm, or 1.5 mm. In some embodiments, the shape of construction element 500 is a cylinder. In other embodiments, the construction element 500 is circular, elliptical, rectilinear, spherical, or hemispherical. In other embodiments construction element 500 has an inner opening in a different shape than its outer opening. In some embodiments, the outer shape of construction element 500 is circular and the inner opening is elliptical or rectilinear. In some embodiments, the construction element has a protrusion that extends from the main to organize wires 510, 520, or wires from another construction element or electrode or electrodes.

FIG. 1D shows a top view of a device 200 assembled in tissue surface 12. In some embodiments, an opening 55 in tissue 50 enables device 200 to enter tissue 10. In some embodiments device 200 has a flexible section 220 that bends out of opening 20. In some embodiments, section 220 has locking features 225 or 230. In some embodiments, the locking components 225 or 230 hold device 200 in position after placement in tissue 10 using a screw 240. In some embodiments it is beneficial to lock section 220 in place to prevent device 200 from excessive motion within tissue. In some embodiments, section 220 can be locked with more precision using mechanical ridges within section 220 and component 225.

In some embodiments, an implanted device can have one or more flexible sections 220 connected to peripheral devices. In some embodiments, the flexible sections 220 are connected to at least one device 100. In still other embodiments, the flexible sections 220 terminate in connectors 240 for connection to peripheral devices. In some embodiments, the connectors 240 connect to at least one device 100.

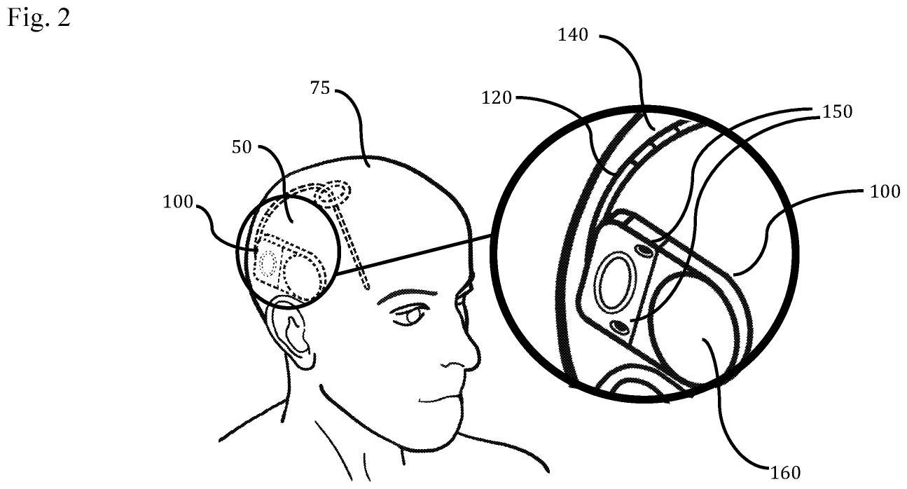

FIG. 2 shows an isometric view of a device 100 assembled to a tissue surface 50 and under a tissue surface 75. In some embodiments, device 100 includes wireless communication. In other embodiments device 100 receives inductive power through a coil. In some embodiments, the device 100 has openings 150 for assembly to tissue. In some embodiments the openings 150 have a diameter between about 0.1 mm and 2.0 mm, such as no more than about 0.2 mm, 0.5 mm, 0.75 mm, 1.0 mm, 1.5 mm, or 1.75 mm. In other embodiments, the device 100 has a thickness between 1.0 mm and 6.0 mm, such as no more than about 1.5 mm, 2.5 mm, 3.5 mm, 4.5 mm, or 5.5 mm. In other embodiments the device 100 has a footprint area between about 10 cm² and 50 cm², such as no more than about 15 cm², 25 cm², 30 cm², 35 cm², 40 cm², or 45 cm².

In some embodiments, an implanted device is slim and compact. In some embodiments, an implanted device can have one or more flexible sections 120 connected to peripheral devices. In some embodiments, the flexible sections are connected to at least one device 200. In still other embodiments, the flexible sections 120 terminate in connectors 140 for connection to peripheral devices. In some embodiments, the connectors 140 connect to at least one device 200. In some embodiments a device 100 that resists fluid infiltration for weeks or months could be implanted and assembled to at least one device 200. In other embodiments, an implanted device 100 that resists fluid infiltration for weeks or months could be disconnected from at least one device 200 and replaced by a device 100 that resists fluid infiltration for years to decades and is connected to at least one still implanted device 200.

In some embodiments, a device 100 can use recording sites of one or more devices 200 to predict and detect tissue activity including epileptic seizures. In some embodiments a device 100 can use recordings from peripheral devices to predict and detect tissue activity including epileptic seizures. In some embodiments, device 100 can stimulate tissue to prevent, inhibit, or end activity including epileptic seizures.

In some embodiments, a device 100 can connect to at least one device 200 and use between about 3 and 1000 electrode sites to record or stimulate, such as no more than about 10, 20, 40, 50, 100, 200, 300, 400, 500, 600, 700, 800, or 900 electrode sites.

In some embodiments device 100 is powered by a battery. In other embodiments, device 100 receives power through an inductive power coil system. In some embodiments, the device 100 communicates with other devices through a radio frequency antenna. In some embodiments, the inductive power coil is contained within zone 160. Any electronic component can be located in any position within device 100. In some embodiments, electrode sites can be located within any zone within device 100.

In some embodiments, connectors 140 are sealed if implanted unconnected to another device. In some embodiments, device 100 and one or more of device 200 are implanted for a period of time to study, diagnose, or treat tissue. In some embodiments, device 100 is ultimately replaced with a longer duration device that is connected to the one or more of device 200. In other embodiments, device 100 is replaced with a longer duration device and some or all of one or more device 200 are replaced with other devices.

In some embodiments, flexible sections 120 connect directly to one or more devices without a connector 140. Implantation is performed by mechanically holding away from tissue all peripheral devices not being implanted. As each peripheral device is implanted, the next peripheral device is released for implantation.

In some embodiments, the device 100 is positioned adjacent the squamous section of the cranium. In other embodiments, the device 100 is positioned adjacent the parietal section of the cranium. In other embodiments, the device 100 is positioned adjacent the occipital section of the cranium.

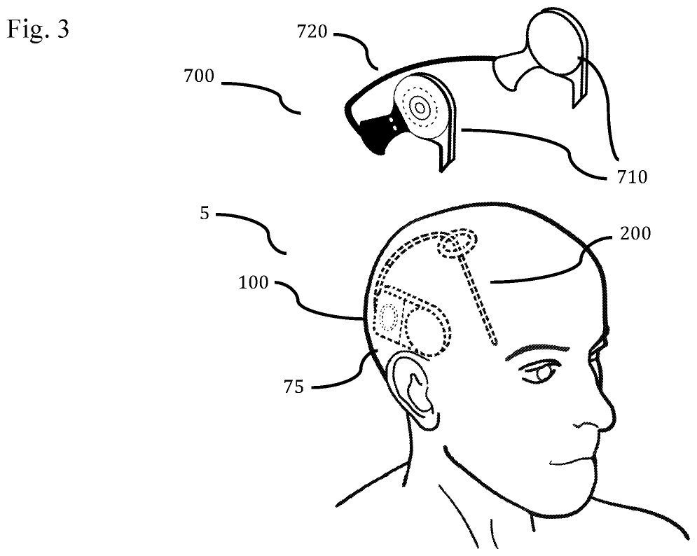

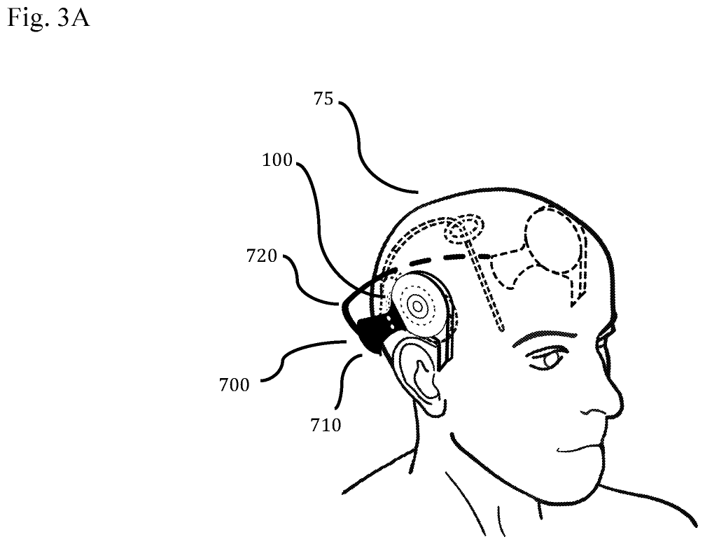

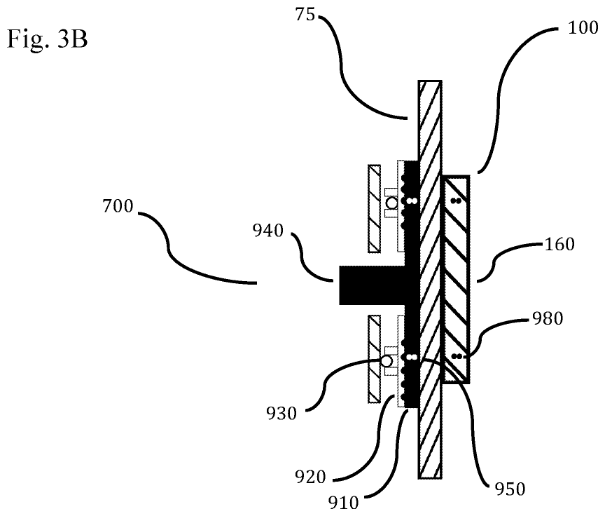

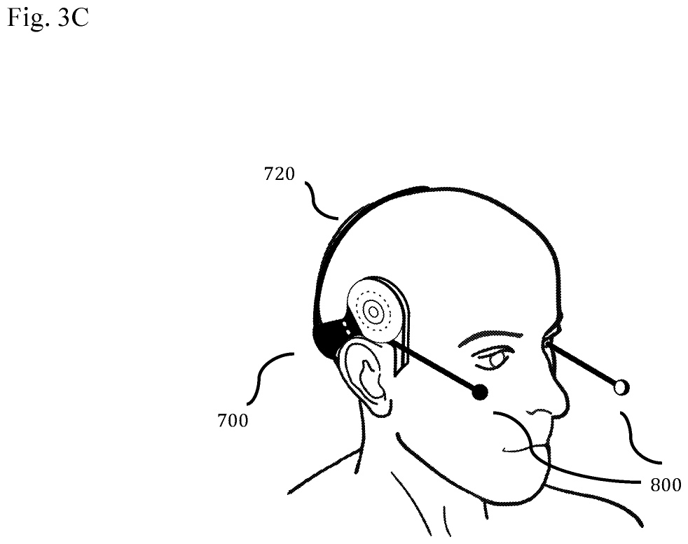

FIG. 3 shows an isometric view of a device 700 and a device system 5 including an implanted device 200 and a device 100. In some embodiments the device 700 is placed on the tissue 75 near one or more device 100. In some embodiments, device 700 terminates in zones 710 that rest adjacent ears. In other embodiments, a zone 710 can be independently positioned to align with a device 100.

FIG. 3A shows an isometric view of a device 700 assembled to tissue 75 to align with an implanted device 100. In some embodiments device 700 is aligned with device 100 with contained magnetic bodies. In other embodiments, wireless communication is used to align the devices. In some embodiments, feedback from backscatter modulation is used to align devices 700 and 100. In some embodiments, feedback from backscatter modulation of the inductive power coils is used to align devices 700 and 100. In other embodiments, the strength of the wireless radio frequency communication is used to align the devices 700 and 100. In other embodiments, infrared light is used from one or more emitters and receivers to align the devices 700 and 100. In some embodiments, lights, sound, vibration, or any other sensory alert is used to assist alignment of devices 700 and 100. In other embodiments, device 700 is aligned with device 100 using mechanical components. In other embodiments, the body 720 is movably assembled to zones 710 to maintain an advantageous alignment. An advantageous alignment can include maintaining the comfort of tissue. An advantageous alignment can also prevent a device from displacing during use. An advantageous alignment can also prevent interference with other devices.

FIG. 3B shows a cross sectional view of device 700 assembled to tissue 75 and aligned with device 100. In some embodiments device 100 is aligned with device 700 using mechanical components 910, and 920. In some embodiments, 910 has protrusions that locate and lock it to component 920. In some embodiments, component 920 has recesses that locate and lock component 910. In some embodiments, a spring 930 forces components 910 and 920 together. In some embodiments zone 940 of component 910 can be displaced to deflect spring 930 reducing the force preventing component 910 from moving relative to component 920. In other embodiments, components 910 and 920 are located and locked together using hook and loop (e.g., Velcro), adhesive, magnets, or any other mechanical fixation. In some embodiments component 910 displaces relative to device 700 using an adhesive, suction, hook and loop (e.g., Velcro), screws, a mechanical detent, a mechanical engagement, press fits, electro-mechanical clamps, a hook, snap fits, jaws, a magnet, friction, zippers, a case, a strap, an elastic member, a cuff, a sleeve, apparel, a thread, a lace, a tie, a rope, a cord, a buckle, a clip, a knot, a loop, a button, or a pocket. In some embodiments, sections of the device 700 are shaped to better conform to an anatomical target. In some embodiments an inductive power coil 950 is within component 910. In other embodiments, an inductive power coil 980 is within zone 160 of device 100. In some embodiments, device 700 is assembled to tissue or clothing using an adhesive, suction, hook and loop (e.g., Velcro), screws, a mechanical detent, a mechanical engagement, press fits, electro-mechanical clamps, a hook, snap fits, jaws, a magnet, friction, zippers, a case, a strap, an elastic member, a cuff, a sleeve, apparel, a thread, a lace, a tie, a rope, a cord, a buckle, a clip, a knot, a loop, a button, or a pocket.

FIG. 3C shows device 700 and body 720 moved to a different position. In some embodiments, a recording device 800 is movably assembled to device 700. In some embodiments, device 800 can wirelessly transmit information to device 700. In other embodiments, device 700 collects recording data including audio, images, video, temperature, position, speed, acceleration, altitude, humidity, time, light emission, and flashing lights. In other embodiments, device 800 collects recording data including audio, images, video, temperature, position, speed, acceleration, altitude, humidity, time, light emission, and flashing lights.

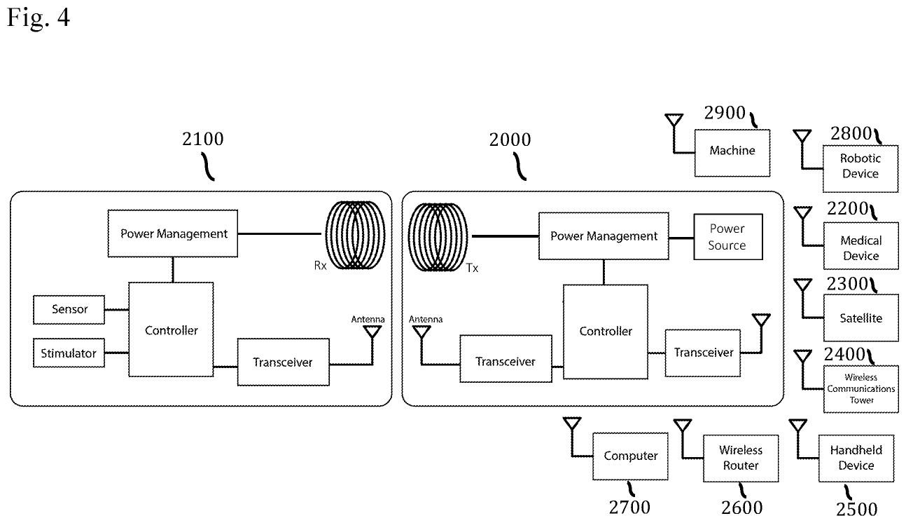

FIG. 4 illustrates a block diagram of a wireless device system. In some embodiments, the device 2000 can operably wirelessly communicate with an implanted device 2100. In some embodiments, the device 2000 can operably transmit power to an implanted device 2100. In some embodiments, the device 2000 can wirelessly communicate with an implanted device 2100 using an inductive power coil. In some embodiments, the device 2000 can operably wirelessly communicate with devices including a computer 2700, a wireless router 2600, a hand-held device 2500, a wireless communication tower 2400, a satellite 2300, a medical device 2200, a robotic device 2800, and a machine 2900. In some embodiments, multiple devices 2000 are used serially for continuous wireless communication with device 2100. In other embodiments, a device 2000 is removed to provide power while another device 2000 establishes wireless communication with device 2100. In some embodiments device 2000 records to a memory storage system. In other embodiments, the memory storage system is removed from device 2000 and communicates with devices including a computer 2700, a wireless router 2600, a hand-held device 2500, a wireless communication tower 2400, a satellite 2300, a medical device 2200, a robotic device 2800, and a machine 2900. In some embodiments the device 2000, a portion of it, or its memory storage system is transported via mail, vehicle, aircraft, boat, robot, or human to a location for analysis of data.

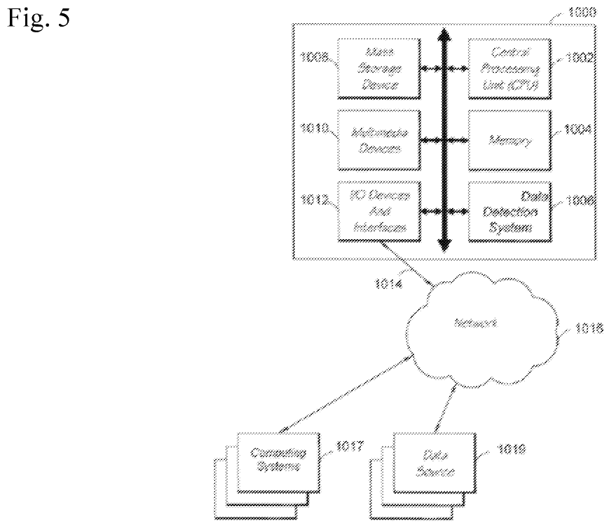

FIG. 5 is a block diagram depicting an embodiment of a computer hardware system configured to run software for implementing one or more embodiments of the systems described herein.

In some embodiments, the computer devices, clients, and/or servers described herein take the form of a computing system 1000 illustrated in FIG. 5 which is a block diagram of one embodiment of a computing system that is in communication with one or more computing systems 1017 and/or one or more data sources 1019 via one or more networks 1016. The computing system 1000 may be used to implement one or more of the systems and methods described herein. In addition, in one embodiment, the computing system 1000 may be configured to manage access or administer a software application. While FIG. 5 illustrates one embodiment of a computing system 1000, it is recognized that the functionality provided for in the components and modules of computing system 1000 may be combined into fewer components and modules or further separated into additional components and modules.

In one embodiment, the computing system 1000 comprises an encoding and/or decoding module 1006 that carries out the functions described herein with reference to detecting and/or processing data, including any one of the techniques described above. The data detection system module 1006 and/or other modules or functional units disclosed herein may be executed on the computing system 1000 by a central processing unit 1002 discussed further below.

In general, the word “module,” as used herein, refers to logic embodied in hardware or firmware, or to a collection of software instructions, possibly having entry and exit points, written in a programming language, such as, for example, COBOL, CICS, Java, Lua, C or C++. A software module may be compiled and linked into an executable program, installed in a dynamic link library, or may be written in an interpreted programming language such as, for example, BASIC, Perl, or Python. It will be appreciated that software modules may be callable from other modules or from themselves, and/or may be invoked in response to detected events or interrupts. Software instructions may be embedded in firmware, such as an EPROM. It will be further appreciated that hardware modules may be comprised of connected logic units, such as gates and flip-flops, and/or may be comprised of programmable units, such as programmable gate arrays or processors. The modules described herein are preferably implemented as software modules, but may be represented in hardware or firmware. Generally, the modules described herein refer to logical modules that may be combined with other modules or divided into sub-modules despite their physical organization or storage.

In one embodiment, the computing system 1000 also comprises a mainframe computer suitable for controlling and/or communicating with large databases, performing high volume transaction processing, and generating reports from large databases. The computing system 1000 also comprises a central processing unit (“CPU”) 1002, which may comprise a conventional microprocessor. The computing system 1000 further comprises a memory 1004, such as random access memory (“RAM”) for temporary storage of information and/or a read only memory (“ROM”) for permanent storage of information, and a mass storage device 1008, such as a hard drive, diskette, or optical media storage device. Typically, the modules of the computing system 1000 are connected to the computer using a standards based bus system. In different embodiments, the standards based bus system could be Peripheral Component Interconnect (PCI), Microchannel, SCSI, Industrial Standard Architecture (ISA), and Extended ISA (EISA) architectures, for example.

The computing system 1000 comprises one or more commonly available input/output (I/O) devices and interfaces 1012, such as a keyboard, mouse, touchpad, and printer. In one embodiment, the I/O devices and interfaces 1012 comprise one or more display devices, such as a monitor, that allows the visual presentation of data to a user. More particularly, a display device provides for the presentation of GUIs, application software data, and multimedia presentations, for example. In one or more embodiments, the I/O devices and interfaces 1012 comprise a microphone and/or motion sensor that allow a user to generate input to the computing system 1000 using sounds, voice, motion, gestures, or the like. In the embodiment of FIG. 10, the I/O devices and interfaces 1012 also provide a communications interface to various external devices. The computing system 1000 may also comprise one or more multimedia devices 1010, such as speakers, video cards, graphics accelerators, and microphones, for example.

The computing system 1000 may run on a variety of computing devices, such as, for example, a server, a Windows server, a Structure Query Language server, a Unix server, a personal computer, a mainframe computer, a laptop computer, a tablet computer, a cell phone, a smartphone, a personal digital assistant, a kiosk, an audio player, an e-reader device, and so forth. The computing system 1000 is generally controlled and coordinated by operating system software, such as z/OS, Windows 95, Windows 98, Windows NT, Windows 2000, Windows XP, Windows Vista, Windows 10, Windows 8, Linux, BSD, SunOS, Solaris, Android, iOS, BlackBerry OS, or other compatible operating systems. In Macintosh systems, the operating system may be any available operating system, such as MAC OS X. In other embodiments, the computing system 1000 may be controlled by a proprietary operating system. Conventional operating systems control and schedule computer processes for execution, perform memory management, provide file system, networking, and I/O services, and provide a user interface, such as a graphical user interface (“GUI”), among other things.

In the embodiment of FIG. 5, the computing system 1000 is coupled to a network 1016, such as a LAN, WAN, or the Internet, for example, via a wired, wireless, or combination of wired and wireless, communication link 1014. The network 1016 communicates with various computing devices and/or other electronic devices via wired or wireless communication links. In the embodiment of FIG. 5, the network 1016 is communicating with one or more computing systems 1017 and/or one or more data sources 1019.

Access to the data detection system module 1006 of the computer system 1000 by computing systems 1017 and/or by data sources 1019 may be through a web-enabled user access point such as the computing systems' 1017 or data source's 1019 personal computer, cellular phone, smartphone, laptop, tablet computer, e-reader device, audio player, or other device capable of connecting to the network 1016. Such a device may have a browser module that is implemented as a module that uses text, graphics, audio, video, and other media to present data and to allow interaction with data via the network 1016. In one embodiment, a removable memory stores data collected by a device implanted within the patient (e.g., device 200, 2100, etc.) and communicated to an external device (e.g., device 100, 2000, etc.). The removable memory may be sent to a caregiver by a user. In another embodiment, usage data may be uploaded to a website accessible by the user and a caregiver. The collected usage data may be used to support various machine learning algorithms (e.g., to train various models, neural networks, etc.), to treat any of a variety of diseases, as a brain-computer interface, such as those described above, may generate enormous amounts of data from multiple patients operating several hours a day (e.g., 24 hours a day), several days a week (e.g., 7 days per week).

The browser module may be implemented as a combination of an all points addressable display such as a cathode-ray tube (CRT), a liquid crystal display (LCD), a plasma display, or other types and/or combinations of displays. In addition, the browser module may be implemented to communicate with input devices 1012 and may also comprise software with the appropriate interfaces which allow a user to access data through the use of stylized screen elements such as, for example, menus, windows, dialog boxes, toolbars, and controls (for example, radio buttons, check boxes, sliding scales, and so forth). Furthermore, the browser module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition a touch screen may act as a hybrid input/output device. In another embodiment, a user may interact with the system more directly such as through a system terminal connected to the score generator without communications over the Internet, a WAN, or LAN, or similar network.

In some embodiments, the system 1000 may comprise a physical or logical connection established between a remote microprocessor and a mainframe host computer for the express purpose of uploading, downloading, or viewing interactive data and databases on-line in real time. The remote microprocessor may be operated by an entity operating the computer system 1000, including the client server systems or the main server system, an/or may be operated by one or more of the data sources 1019 and/or one or more of the computing systems 1017. In some embodiments, terminal emulation software may be used on the microprocessor for participating in the micro-mainframe link.

In some embodiments, computing systems 1017 who are internal to an entity operating the computer system 1000 may access the data detection system module 1006 internally as an application or process run by the CPU 1002.

In an embodiment, a user access point or user interface comprises a personal computer, a laptop computer, a tablet computer, an e-reader device, a cellular phone, a smartphone, a GPS system, a Blackberry® device, a portable computing device, a server, a computer workstation, a local area network of individual computers, an interactive kiosk, a personal digital assistant, an interactive wireless communications device, a handheld computer, an embedded computing device, an audio player, a smartphone, a smartwatch, or the like.

In addition to the systems that are illustrated in FIG. 5, the network 1016 may communicate with other data sources or other computing devices. The computing system 1000 may also comprise one or more internal and/or external data sources. In some embodiments, one or more of the data repositories and the data sources may be implemented using a relational database, such as DB2, Sybase, Oracle, CodeBase and Microsoft® SQL Server as well as other types of databases such as, for example, a flat file database, an entity-relationship database, and object-oriented database, and/or a record-based database.

Although certain embodiments of the disclosure have been described in detail, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. In addition, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described above. For all of the embodiments described above, the steps of any methods need not be performed sequentially. The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

Other Considerations

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

Many other variations than those described herein will be apparent from this disclosure. For example, depending on the embodiment, certain acts, events, or functions of any of the algorithms described herein can be performed in a different sequence, can be added, merged, or left out altogether (e.g., not all described acts or events are necessary for the practice of the algorithms). Moreover, in certain embodiments, acts or events can be performed concurrently, e.g., through multi-threaded processing, interrupt processing, or multiple processors or processor cores or on other parallel architectures, rather than sequentially. In addition, different tasks or processes can be performed by different machines and/or computing systems that can function together.

The various illustrative logical blocks, modules, and algorithm elements described in connection with the embodiments disclosed herein can be implemented as electronic hardware, computer software, or combinations of both. To clearly illustrate this interchangeability of hardware and software, various illustrative components, blocks, modules, and elements have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the embodiments disclosed herein can be implemented or performed by a machine, such as a processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, or algorithm described in connection with the embodiments disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC. The ASIC can reside in a user terminal. In the alternative, the processor and the storage medium can reside as discrete components in a user terminal.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements, and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Unless otherwise explicitly stated, articles such as “a”, “an”, or “the” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B, and C” can include a first processor configured to carry out recitation A working in conjunction with a second processor configured to carry out recitations B and C.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, certain embodiments described herein can be implemented within a form that does not provide all of the features and benefits set forth herein, as some features can be used or practiced separately from others. All such modifications and variations are intended to be included herein within the scope of this disclosure. Further, additional embodiments created by combining any two or more features or techniques of one or more embodiments described herein are also intended to be included herein within the scope of this disclosure.

What is claimed is:

1. A method of treating a neurological condition in a medical patient, comprising:
implanting a first set of leads at a first target tissue site within target tissue of the medical patient, wherein each lead comprises one electrode or more than one electrodes spaced apart from each other along a length of each lead of the first set of leads;
implanting a second set of leads at a second target tissue site within the target tissue of the medical patient, wherein each lead comprises one electrode or more than one electrodes spaced apart from each other along a length of each lead of the second set of leads;
receiving a first signal via the first set of leads, the first signal indicative of electrical activity at the first target tissue site during an event;
receiving a second signal via the second set of leads, the second signal indicative of electrical activity at the second target tissue site during the event;
determining that a parameter of the first signal satisfies a condition;
determining that a parameter of the second signal does not satisfy the condition; and
removing only the second set of leads from the medical patient.

2. The method of claim 1, further comprising sending a stimulating signal to the first target tissue site via the first set of leads.

3. The method of claim 1, wherein receiving the first signal via the first lead comprises receiving the first signal via the first lead continuously or intermittently over a predetermined data collection period of minutes, hours, days, weeks, or months.

4. The method of claim 1, wherein the target tissue comprises brain tissue, nerve tissue, cardiac tissue, or bone.

5. The method of claim 1, wherein the parameter of the first signal comprises a Voltage level, a current level, a frequency, or an impedance.

6. The method of claim 1, wherein the first set of leads comprises a single lead, about 10 leads, about 30 leads, about 50 leads, about 100 leads, about 1000 leads, about 2,500 leads, or more than 1000 leads.

7. The method of claim 1, wherein implanting the first set of leads at the first target tissue site comprises implanting the first set of leads at the first tissue site at a first depth, wherein implanting the second set of leads at the second target tissue site comprises implanting the second set of leads at a second depth within the target tissue, and wherein the first depth is different than the second depth.

8. The method of claim 1, wherein the event comprises electrical activity related to a seizure, an epileptic seizure, seizure onset, therapy resistant depression, vision restoration, hearing restoration, speech restoration, motor control restoration, sleep dysfunction, or Alzheimer's disease.

9. The method of claim 1, wherein the first set of leads comprises a wireless transmitter or transceiver configured to wirelessly transmit the first signal to a wireless receiver or transceiver.

10. The method of claim 1, wherein receiving the first signal comprises wirelessly receiving the first signal from a wireless transmitter coupled to the first set of leads.

11. The method of claim 1, further comprising resecting at least a portion of the target tissue at the first or second target tissue site.

12. The method of claim 1, wherein determining that a parameter of the first signal satisfies a condition comprises utilizing a machine learning algorithm, deep learning, a neural network, or artificial intelligence.

13. The method of claim 1, further comprising determining a stimulation based upon a detected condition utilizing a machine learning algorithm, deep learning, a neural network, or artificial intelligence.

14. A medical headset configured to treat a neurological condition in a medical patient, comprising:
a headgear configured to be worn on the head of a medical patient and configured to be positioned or located with respect to an implant without the use of magnets;
a wireless receiver or transceiver supported by the headgear and configured to receive a wireless signal from a wireless transmitter coupled to a first set of leads implanted at a first target tissue site within target tissue of the medical patient;
a power source, supported by the headgear and configured to: (i) provide power to the wireless receiver or transceiver, and (ii) wirelessly provide power to the implant;
a processor, coupled to the wireless receiver and configured to store neural activity data corresponding to the wireless signal in a data store; and
the data store.

15. The medical headset of claim 14, further comprising an interface for transferring the neural activity data to a computing system.

16. The medical headset of claim 15, wherein the interface comprises a USB port, a wireless transceiver, a SIM slot, or a memory card slot.

17. The medical headset of claim 14, wherein the power source comprises a battery.

18. The medical headset of claim 14, wherein the data store comprises a removable memory device configured to be removed from the medical headset and transported to a caregiver, or a machine learning algorithm developer.

19. The medical headset of claim 14, wherein the medical headset further comprises an adjustable mechanical engagement of an applied force source and mechanical detents in at least two dimensions, wherein the adjustable mechanical engagement enables fine tuning and adjustment of the position of the wireless receiver or transceiver with respect to an implanted wireless transmitter or transceiver of the implant.

20. The medical headset of claim 14, further comprising a sensor, the sensor comprising one or more of a camera, an accelerometer, a thermometer, a humidity sensor, a perspiration sensor, a gyroscope, a switch, a microphone, a light sensor, or a GPS, wherein the sensor is configured to generate a signal associated with or an image of the medical patient, and wherein the processor is further configured to determine a clinical event based on the signal.
